(19) United States
(12) Patent Application Publication
(10) Pub. No.: US 2025/0387787 A1
(43) Pub. Date: Dec. 25, 2025

(54) METHOD AND SYSTEM FOR CONSTRUCTING SENSOR SURFACE AND USAGE THEREOF

(71) Applicant: Stichting IMEC Nederland, Wageningen (NL)

(72) Inventors: Arjan Tibbe, Deventer (NL); Adriana Karcz, Veldhoven (NL)

(21) Appl. No.: 19/230,824

(22) Filed: Jun. 6, 2025

(30) Foreign Application Priority Data
Jun. 25, 2024 (EP) 24184161.8

(51) Int. Cl. B01L 3/00

(52) U.S. Cl. CPC B01L 3/502707; B01L 3/502715; B01L 2200/028; B01L 2300/0645; B01L 2300/0663; B01L 2300/0681; B01L 2400/0487

(57) ABSTRACT

A method and system for constructing a sensor surface is provided. The method may method include providing a buffer fluid comprising at least one sensing particle. The method may further include providing a membrane comprising a plurality of pores, each of the plurality of pores having a pore size smaller than the sensing particle. The method may also include arranging the membrane in relation to the buffer fluid such that a first surface of the membrane being in fluidic contact with the buffer fluid. Further, the method may include pushing the buffer fluid through the membrane to immobilize the sensing particle on the first surface of the membrane.

### CROSS-REFERENCE TO RELATED APPLICATIONS

The present application is a non-provisional patent application claiming priority to Application No EP 24184161.8, filed Jun. 25, 2024, the contents of which are hereby incorporated by reference.

### FIELD OF THE DISCLOSURE

The disclosure relates to the construction of sensor surfaces, such as whole-cell biosensor surfaces, and further to the use of the sensor surfaces, such as in the form of whole-cell biosensors, for detecting and/or measuring biological and/or chemical activities.

### BACKGROUND

Generally, whole-cell sensors can sense compounds of interests, i.e., analytes, in complex fluids and matrices that contain a variety of molecules and particles, e.g., the real time detection and concentration measurements of specific proteins inside bioreactors. The general concept of whole-cell biosensors is that living cells convert inputs which are otherwise difficult to measure, e.g., metabolites, chemicals, cytokines, hormones, as well as other compounds that cannot be measured with existing biosensors, into the measurable physical parameters.

For example, the presence of a certain protein can be specifically detected by the cell that, in turn, generates a physical signal, such as light or charge, which can be detected by conventional detectors. It may be beneficial for the instructions for the cell sensing, processing and actuation abilities to be embedded in the DNA of the cell using different biotechnological, biosynthetic, and genetic engineering methods.

Porous membranes may be used for cell cultures, such as in cellular co-culture systems. However, avoidance of mixing of the genetically engineered sensing with other cells may need to be taken into consideration. This may be not considered in the co-culture systems and in the field of genetically engineered sensing.

### SUMMARY

Current methodologies for building biosensor surfaces may rely on printing technologies in combination with surface chemistries to fix and mount the biosensors on the sensor surface. Additionally, in order to enable successful printing, the fluids in which the biosensors reside may need to match the printer requirements as well as the properties of the material of the sensor surface, such as hydrophobicity among others.

Example embodiments described herein provide a method and a system for constructing a sensor surface in a simplified manner in order to overcome the above-mentioned limitations. Example embodiments further describe a sensor for detecting and/or measuring biological and/or chemical activities in a simplified and cost-effective manner.

These and other objects may be addressed by the features of the first independent claim for the method, by the features of the second independent claims for the system, and by the features of the third independent claim for the sensor. The dependent claims contain further developments.

According to a first aspect of the disclosure, a method for constructing a sensor surface is provided. The method may comprise a step of providing a buffer fluid comprising at least one sensing particle. In addition, the method may further comprise a step of providing a membrane comprising a plurality of pores, each of the plurality of pores has a pore size smaller than the sensing particle. In addition, the method may comprise a step of arranging the membrane in relation to the buffer fluid such that a first surface of the membrane being in fluidic contact with the buffer fluid. Moreover, the method may comprise a step of pushing the buffer fluid through the membrane to immobilize and/or to concentrate the sensing particle on the first surface of the membrane.

Therefore, a physical confinement may be facilitated by separating the sensing particles, such as whole-cell biosensor cells, from the sample fluid, which may form a sensor surface, such as a whole-cell biosensor surface, in a simplified manner.

One potential advantage may be, by using the porous membrane to immobilize the sensing particles to form the sensor surface, the sensing particles may be suspended in any type of fluid independently on the surface properties or printer requirements. Furthermore, the sensing particles can be concentrated on the sensing surface. Moreover, the sensing particles may not be able to escape from the sensing surface, which may eliminate further application of chemical crosslinking and surface chemistry.

According to some example embodiments, the sensing particle may be configured to produce light and/or to change color and/or to change charge and/or to produce gases and/or to change in size and/or to produce at least one measurable change in reaction to sample analytes. For instance, the sensing particles may be hydrogel beads synthesized with a glucose sensitive dye designed to sense glucose.

According to some example embodiments, the sensing particle may comprise at least one whole-cell eukaryotic cell or prokaryotic cell, or at least one non-whole cell particle containing one or more whole-cell components, or at least one hydrogel particle, or at least one polystyrene particle, or at least one ceramic particle, or a combination thereof.

It may be noted that the sensing particle may not be limited to the above-mentioned hydrogel, polystyrene, and ceramic materials. Other types of suitable materials can be used to form or to create the sensing particle.

According to some example embodiments, the membrane may contain a plurality of pores having a pore size between 0.1 to 0.5 microns, such as a pore size between 0.2 to 0.3 microns or a pore size between 0.20 to 0.25 microns.

According to a second aspect of the disclosure, a system for constructing a sensor surface is provided. The system may comprise at least one pumping arrangement comprising a buffer fluid comprising at least one sensing particle, the pumping arrangement being configured to push the buffer fluid through an opening of the pumping arrangement. In addition, the system may comprise at least one filter arrangement comprising a membrane comprising a plurality of pores, each of the plurality of pores having a pore size smaller than the sensing particle.

The filter arrangement may be detachably attached to the opening of the pumping arrangement such that a first surface of the membrane being in fluidic contact with the buffer fluid. Furthermore, the membrane may be configured to immobilize and/or to concentrate the sensing particle on the first surface while the buffer fluid is pushed through the membrane by the pumping arrangement.

Therefore, a physical confinement may be facilitated by separating the sensing particles, such as whole-cell biosensor cells, from the sample fluid, which may form a sensor surface, such as a whole-cell biosensor surface, in a simplified manner.

According to some example embodiments, the system may comprise at least one further pumping arrangement comprising a further buffer fluid comprising at least one further sensing particle. In some example embodiments, the system may comprise at least one further filter arrangement comprising a further membrane comprising a further plurality of pores, each of the further plurality of pores having a pore size smaller than the further sensing particle.

The further membrane may be configured to immobilize and/or to concentrate the further sensing particle on a first surface of the further membrane while the further buffer fluid is pushed through the further membrane by the further pumping arrangement.

A potential benefit of embodiments described herein may be that a plurality of sensor surfaces, such as whole-cell biosensor surfaces, can be formed in a simplified manner, which can perform parallel sensing of a plurality of analytes, respectively.

The membrane may comprise a plurality of pores having a pore size between 0.1 to 0.5 microns, such as a pore size between 0.2 to 0.3 microns or a pore size between 0.20 to 0.25 microns.

The further membrane may comprise a further plurality of pores having a pore size between 0.1 to 0.5 microns, such as a pore size between 0.2 to 0.3 microns or a pore size between 0.20 to 0.25 microns.

The pumping arrangement and/or the further pumping arrangement may be syringes or have a syringe-like configuration. For example, the fluid delivery mechanism may be realized in a simplified manner with high precision and control.

The filter arrangement and/or the further filter arrangement may be syringe filters or filters having components of a syringe. For example, the filter arrangement may comprise or be a syringe filter with a pore size of 0.22 microns. As another example, the filter arrangement may comprise or be a syringe filter with a pore size of 0.45 microns.

According to a third aspect of the disclosure, a sensor is provided. The sensor may comprise at least one filter arrangement comprising a membrane, and at least one sensing particle immobilized on a first surface of the membrane. The sensor may further comprise a readout module being arranged in relation to the first surface of the membrane.

The filter arrangement may be configured to pass analytes onto a second surface of the membrane opposite to the first surface in order for the analytes to diffuse through the membrane. Furthermore, the readout module may be configured to detect a property of the sensing particle in reaction to the analytes diffused through the membrane.

A potential application of the sensor surface, i.e., the sensor surface that may be constructed by separating the sensing particles from the sample fluid, is provided by means of sensor particles, such as whole-cell biosensors, which may detect and/or measure biological and/or chemical activities in reaction to the analytes.

The filter arrangement may be detachably attached to the readout module. For example, the readout module may be detached from one filter arrangement, and may be re-used for another filter arrangement.

The readout module may comprise an optical detector configured to detect an optical property of the sensing particle in reaction to the analytes diffused through the membrane.

The readout module may comprise an electrical signal detector configured to detect an electrical property of the sensing particle in reaction to the analytes diffused through the membrane.

The readout module may comprise a detector unit configured to simultaneously detect an optical property and an electrical property of the sensing particle in reaction to the analytes diffused through the membrane.

The filter arrangement may comprise a first channel being in fluidic contact with the first surface of the membrane, and a second channel being in fluidic contact with the second surface of the membrane. The first channel may be configured to confine the sensing particle immobilized on the first surface of the membrane and/or the analytes diffused through the membrane.

A fluidic isolation between the filter arrangement and the readout module may be facilitated. Furthermore, the second channel may be configured to pass the analytes onto the second surface of the membrane for the analytes to diffuse through the membrane.

The filter arrangement and/or the detector unit may be sterilizable. For example, the sensor may be effectively used in bio-controlled environments.

It is to be noted that the system according to the second aspect may correspond to the method according to the first aspect and its implementation forms. It is further to be noted that the elements and/or components of the sensor according to the third aspect may have corresponding implementation forms of the analogous elements and/or components according to the method of the first aspect and/or the system of the second aspect.

### BRIEF DESCRIPTION OF THE FIGURES

The above, as well as additional, features will be better understood through the following illustrative and non-limiting detailed description of example embodiments, with reference to the appended drawings.

Exemplary embodiments of the disclosure are now further explained with respect to the drawings by way of example only, and not for limitation. In the drawings:

FIG. 1 shows an exemplary embodiment of a method

FIG. 2 shows a first exemplary embodiment of a system.

FIG. 3 shows a second exemplary embodiment of a system.

FIG. 4 shows a third exemplary embodiment of a system.

FIG. 5 shows a first exemplary embodiment of a sensor.

FIG. 6 shows a second exemplary embodiment of a sensor.

FIG. 7 shows a first exemplary embodiment of a readout module.

FIG. 8 shows a second exemplary embodiment of a readout module.

FIG. 9 shows a third exemplary embodiment of a readout module.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

Reference will now be made in detail to embodiments of the present disclosure, examples of which are illustrated in the accompanying drawings. However, the following embodiments may be variously modified and the range of the present disclosure is not limited by the following embodiments.

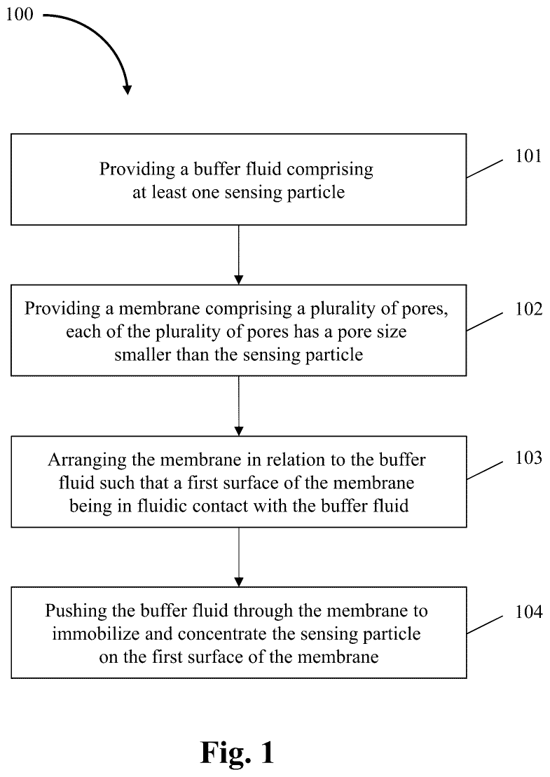

FIG. 1 illustrates an exemplary embodiment of the method 100. Step 101 may include providing a buffer fluid comprising at least one sensing particle. Step 102 may include providing a membrane comprising a plurality of pores is provided, where each pore of the plurality of pores has a pore size smaller than the sensing particle.

The method may include step 103 that may include arranging the membrane in relation to the buffer fluid such that a first surface of the membrane being in fluidic contact with the buffer fluid. The method may further include step 104, which may include pushing the buffer fluid through the membrane to immobilize and concentrate the sensing particle on the first surface of the membrane.

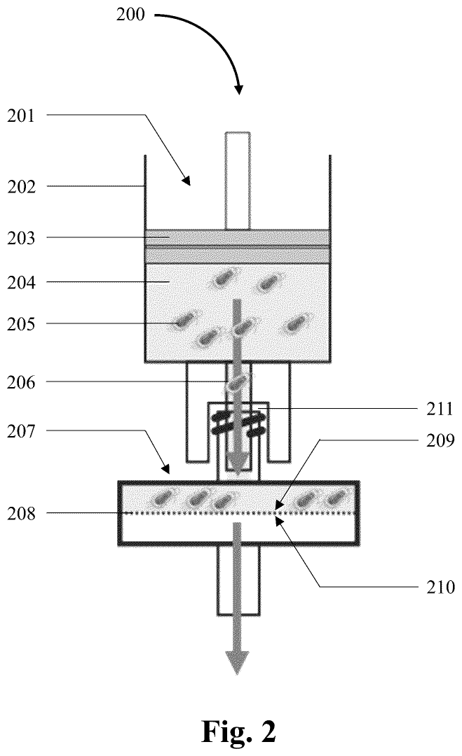

FIG. 2 shows a first exemplary embodiment of a system 200. The system 200 may comprise a pumping arrangement 201, such as a syringe, comprising a barrel 202, a plunger 203 that is tightly fitted to the inside of the barrel 202, and an opening 206. The plunger 203 can be linearly pushed along the inside of the barrel 202 towards the opening 206.

The pumping arrangement 201 may contain buffer fluid 204 comprising sensing particles 205 or sensing cells that may be confined inside of the barrel 202 between the plunger 203 and the opening 206.

The system 200 may further comprise a filter arrangement 207, such as a syringe filter, comprising a porous membrane 208, such as a microporous membrane, with pore size smaller than the size of the sensing particles 205 in the buffer fluid 204. The filter arrangement 207 may be detachably attached to the pumping arrangement 201 via a locking mechanism 211, such as a luer-taper or a luer-lock fluidic fitting, such that a first surface 209 of the porous membrane 208 being in fluidic contact with the opening 206.

The buffer fluid 204 may be pushed by the pumping arrangement 201, such as by the plunger 203, through the opening 206, and further through the porous membrane 208. Since the porous membrane 208 may have pore size smaller than the sensing particles 205, only the buffer fluid 204 may be passed through the porous membrane 208 towards a second surface 210 of the porous membrane 208, leaving behind the sensing particles 205 immobilized on the first surface 209.

The bold arrow shows the flow direction of the buffer fluid 204 through the opening 206 and further through the porous membrane 208, such as due to the force applied by the plunger 203 of the pumping arrangement 201. The filter arrangement 207 can be detached from the pumping arrangement 201, and the first surface 209 of the porous membrane 208 with the immobilized sensing particles 205 can be used as a sensing surface, such as a biosensor surface.

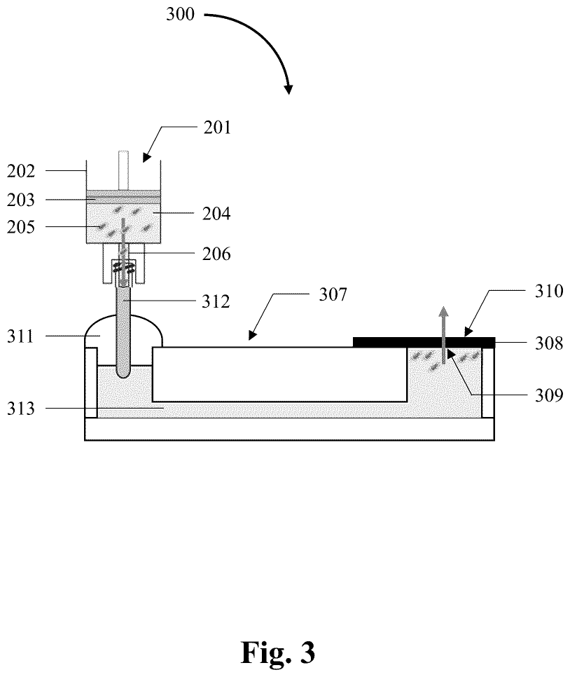

FIG. 3 shows a second exemplary embodiment of a system 300. The system 300 may comprise a pumping arrangement 201 analogous to the system 200, i.e., comprising a barrel 202, a plunger 203, and an opening 206, where the barrel may contain a buffer fluid 204 comprising sensing particles 205.

The system 300 may further comprise a filter arrangement 307 comprising a porous membrane 308, such as a microporous membrane, with pore size smaller than the size of the sensing particles 205 in the buffer fluid 204. The filter arrangement 307 may comprise a fluidic channel 313 being in fluidic contact with a first surface 309 of the porous membrane 308.

For instance, the filter arrangement 307 may be detachably attached to the pumping arrangement 201 via a locking mechanism, such as a rubber cap or stopper 311 arranged at an opening of the fluidic channel 313 and a needle 312 arranged at the opening 206 of the pumping arrangement 201.

The buffer fluid 204 may be pushed through the rubber cap or stopper into the fluidic channel 313 and the pumping arrangement 201 may be detached easily when needed. This may cause the first surface 209 of the porous membrane 208 to be in fluidic contact with the opening 206 of the pumping arrangement 201 via the fluidic channel 313.

The buffer fluid 204 may be pushed by the pumping arrangement 201, such as by the plunger 203, through the opening 206, along the fluidic channel 313, and further through the porous membrane 308. Since the porous membrane 308 may have pore size smaller than the sensing particles 205, only the buffer fluid 204 may be passed through the porous membrane 308 towards a second surface 310 of the porous membrane 308, leaving behind the sensing particles 205 immobilized on the first surface 309.

The bold arrow shows the flow direction of the buffer fluid 204 through the opening 206 and further through the porous membrane 308, such as due to the force applied by the plunger 203 of the pumping arrangement 201. The filter arrangement 307 can be detached from the pumping arrangement 201, and the first surface 309 of the porous membrane 308 with the immobilized sensing particles 205 can be used as a sensing surface, for example as a biosensor surface.

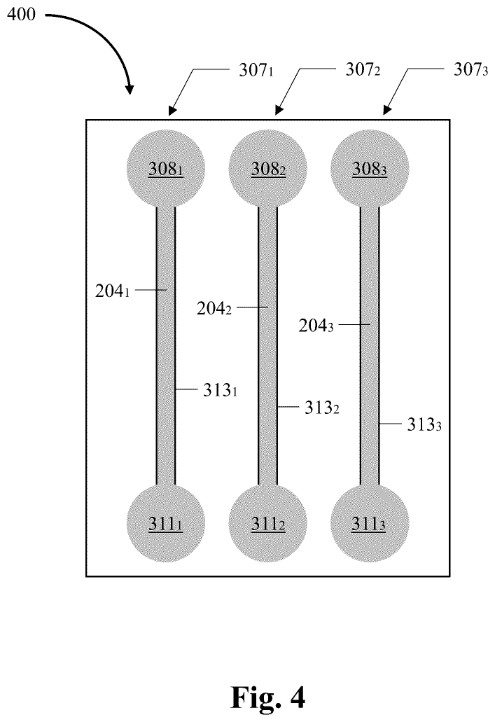

FIG. 4 shows a third exemplary embodiment of a system 400. The system 400 may comprise a plurality of filter arrangements 307, exemplary a first filter arrangement 307₁, a second filter arrangement 307₂, and a third filter arrangement 307₃. However, the number of the filter arrangements can be more than or less than three, as illustrated herein. For example, each of the filter arrangements 307₁-307₃ may correspond to the filter arrangement 307 of the system 300.

The first filter arrangement 307₁ may comprise a porous membrane 308₁ with pore size smaller than the size of the sensing particles 205 in a first buffer fluid 204₁, a fluidic channel 313₁ being in fluidic contact with a first surface of the porous membrane 308₁, and a locking mechanism 311₁. The first buffer fluid 204₁ may be pushed by a first pumping arrangement (not shown) along the fluidic channel 313₁, and further through the porous membrane 308₁, which may immobilize the sensing particles 205 on the first surface of the porous membrane 308₁.

The second filter arrangement 307₂ may comprise a porous membrane 308₂ with pore size smaller than the size of the sensing particles 205 in a second buffer fluid 204₂, a fluidic channel 313₂ being in fluidic contact with a first surface of the porous membrane 308₂, and a locking mechanism 311₂. The second buffer fluid 204₂ may be pushed by a second pumping arrangement (not shown) along the fluidic channel 313₂, and further through the porous membrane 308₂, which may immobilize the sensing particles 205 on the first surface of the porous membrane 308₂.

The third filter arrangement 307₃ may comprise a porous membrane 308₃ with pore size smaller than the size of the sensing particles 205 in a third buffer fluid 204₃, a fluidic channel 313₃ being in fluidic contact with a first surface of the porous membrane 308₃, and a locking mechanism 311₃. The third buffer fluid 204₃ may be pushed by a third pumping arrangement (not shown) along the fluidic channel 313₃, and further through the porous membrane 308₃, which may immobilize the sensing particles 205 on the first surface of the porous membrane 308₃.

It is to be noted, each of the first pumping arrangement, the second pumping arrangement, and the third pumping arrangement may correspond to the pumping arrangement 201 of the system 200. Furthermore, each of the first buffer fluid 204₁, the second buffer fluid 204₂, and the third buffer fluid 204₃ may correspond to the buffer fluid 204 of the system 200.

The first buffer fluid 204₁, the second buffer fluid 204₂, and the third buffer fluid 204₃ may be different from each other, i.e., comprising different sensing particles that may produce different measurable changes in reaction to different sample analytes.

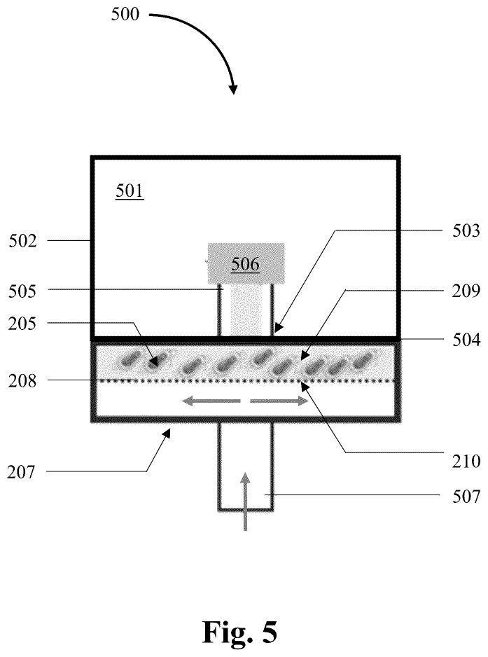

FIG. 5 shows a first exemplary embodiment of a sensor 500. The sensor 500 may comprise a filter arrangement 207, which may correspond to the filter arrangement 207 of the system 200, where the sensing particles 205 are immobilized on the first surface 209 of the porous membrane 208.

The sensor 500 may further comprise a readout module 501 arranged in relation to the first surface 209 of the porous membrane 208. The readout module 501 may comprise a housing 502, and an aperture 503 such as arranged at an attachment surface 504 of the housing 502.

The filter arrangement 207 may comprise a first channel 505 being in fluidic contact with the first surface 209 of the porous membrane 208, and a second channel 507 being in fluidic contact with the second surface 210 of the porous membrane 208.

To attach the filter arrangement 207 to the readout module 501, the first channel 505 of the filter arrangement 207 may be positioned inside of the housing 502 of the readout module 501, such as through the aperture 503 at the attachment surface 504, and may be fixed at the attachment surface 504, e.g., using an adhesive substance. Therefore, the filter arrangement 207 may be detached from the readout module 501 if needed.

To provide a fluidic isolation between the filter arrangement 207 and the readout module 501, the first channel 505 of the filter arrangement 207 may comprise a locking mechanism 506, e.g., a luer-lock, which may restrict the sensing particles 205 and any remaining buffer fluid 204 within the filter arrangement 207, such as in the first channel 505.

The second channel 507 of the filter arrangement 207 may allow sample fluids comprising compounds of interest, e.g., bioreactor sample fluids comprising analytes, to pass onto the second surface 210 of the porous membrane 208 for the analytes to be diffused through the porous membrane 208. The bold arrow shows the flow direction of a sample fluid through the second channel 507 of the filter arrangement 207 onto the second surface 210 of the porous membrane 208.

The readout module 501 may detect a property of the sensing particles 205, e.g., one or more optical properties and/or one or more electrical properties of the sensing particles 205, in reaction to the analytes diffused through the porous membrane 208.

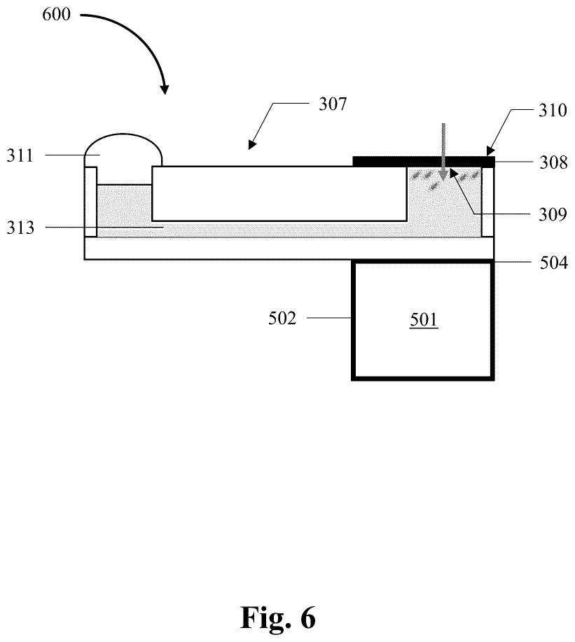

FIG. 6 shows a second exemplary embodiment of a sensor 600. The sensor 600 may comprise a filter arrangement 307, which may correspond to the filter arrangement 307 of the system 300, where the sensing particles 205 are immobilized on the first surface 309 of the porous membrane 308.

The sensor 600 may further comprise a readout module 501 arranged in relation to the first surface 309 of the porous membrane 308. The readout module 501 may correspond to the readout module 501 of the sensor 500. In order to attach the filter arrangement 307 to the readout module 501, the filter arrangement 307 may be fixed at the attachment surface 504 of the readout module 501, e.g., using an adhesive substance. Therefore, the filter arrangement 307 can be detached from the readout module 501 if needed.

For instance, the filter arrangement 307 may allow sample fluids comprising compounds of interest, e.g., bioreactor sample fluids comprising analytes, to pass onto the second surface 310 of the porous membrane 308 for the analytes to be diffused through the porous membrane 308. The bold arrow shows the diffusion of analytes from the second surface 310 to the first surface 309 through the porous membrane 308.

The readout module 501 may detect a property of the sensing particles 205, e.g., one or more optical properties and/or one or more electrical properties of the sensing particles 205, in reaction to the analytes diffused through the porous membrane 308.

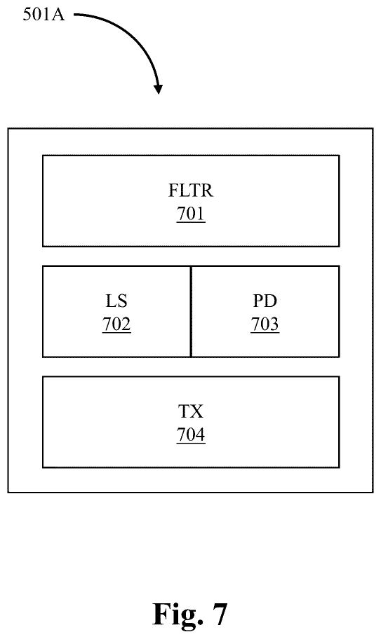

FIG. 7 shows a first exemplary embodiment of a readout module 501A. The readout module 501A may comprise or be an optical detector and may detect an optical property of the sensing particle 205, e.g., a light signal produced and/or a change in color in reaction to the analytes diffused through the membrane 208, 308.

The readout module 501A may comprise a filter or grating structure 701 configured for an excitation light signal of a predefined wavelength and further for an emission light signal of a predefined wavelength. The readout module 501A may further comprise a light source 702, such as a light-emitting diode, which may generate the excitation light signal to illuminate the sensing particle 205 such as through the filter structure 701, e.g., to achieve the excited state of the sensing particle 205.

The readout module 501A may further comprise a photodetector 703, such as a photodiode, which may receive the emission light signal from the sensing particle 205, such as through the filter structure 701. The readout module 501A may further comprise a transmitter 704 that may receive the optical measurements from the photodetector 703, e.g., the photodiode currents or voltages, and may transmit the measurements to an external device, such as wirelessly, for further pursing and/or processing of the measurement data.

The readout module 501A may additionally comprise a processor (not shown) that may perform pre-processing of the optical measurements before sending out the measurements through the transmitter 704. In addition, the readout module 501A may comprise a power source (not shown), e.g., a battery, in order to provide electric power to the components of the readout module 501A.

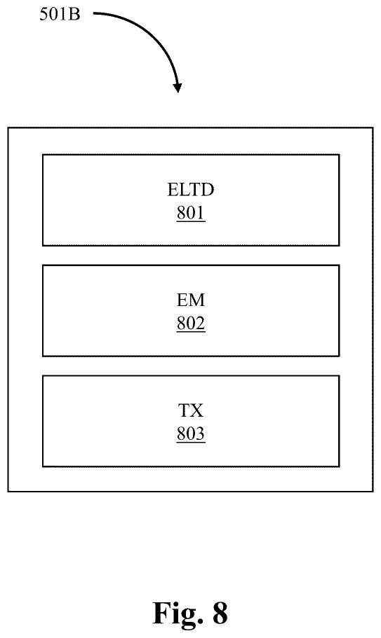

FIG. 8 shows a second exemplary embodiment of a readout module 501B. The readout module 501A may comprise or be an electrical signal detector and may detect an electrical property of the sensing particle 205, e.g., the production and/or change in charge in reaction to the analytes diffused through the membrane 208, 308.

The readout module 501B may comprise an electrode 801 configured to be in electrically coupled to the sensing particle 205. The detector 501B may further comprise an electrometer 802, such as a potentiometer, electrically coupled to the electrode 801. The electrometer 802 may measure an electrical signal produced by the sensing particle 205.

The detector 501B may further comprise a transmitter 803 that may receive the electrical measurements from the electrometer 802 and may transmit the measurements to an external device, such as wirelessly, for further pursing and/or processing of the measurement data.

The readout module 501B may additionally comprise a processor (not shown) that may perform pre-processing of the electrical measurements before sending out the measurements through the transmitter 803. In addition, the readout module 501B may comprise a power source (not shown), e.g., a battery, in order to provide electric power to the components of the readout module 501B.

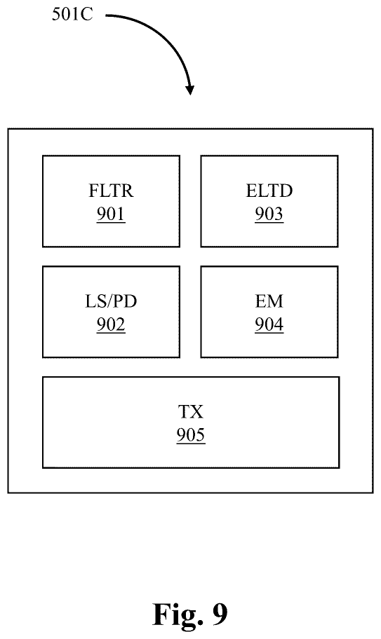

FIG. 9 shows a third exemplary embodiment of a readout module 501C. The readout module 501A may comprise or be a detector unit or a hybrid detector and may detect an optical property of the sensing particle 205, e.g., a light signal produced and/or a change in color, as well as an electrical property of the sensing particle 205, e.g., the production and/or change in charge, such as in reaction to the analytes diffused through the membrane 208, 308.

The readout module 501C may comprise a filter or grating structure 901 configured for an excitation light signal of a predefined wavelength and further for an emission light signal of a predefined wavelength. The readout module 501C may further comprise a light detection unit 902, such as comprising a light-emitting diode and a photodiode, which may generate the excitation light signal and may receive the emission light signal from the sensing particle 205, such as through the filter structure 901.

In addition, the readout module 501C may comprise an electrode 903 configured to be in electrically coupled to the sensing particle 205, and an electrometer 904, such as a potentiometer, electrically coupled to the electrode 903. The electrometer 904 may measure an electrical signal produced by the sensing particle 205.

The readout module 501C may further comprise a transmitter 905 that may receive the optical measurements from the light detection unit 902 and the electrical measurements from the electrometer 904, and may transmit the measurements to an external device, such as wirelessly, for further pursing and/or processing of the measurement data.

The readout module 501C may additionally comprise a processor (not shown) that may perform pre-processing of the optical measurements and the electrical measurements before sending out the measurements through the transmitter 905. In addition, the readout module 501C may comprise a power source (not shown), e.g., a battery, in order to provide electric power to the components of the readout module 501C.

In the description as well as in the claims, the word “comprising” does not exclude other elements or steps and the indefinite article “a” or “an” does not exclude a plurality. A single element or other unit may fulfill the functions of several entities or items recited in the claims.

The term “and/or” used in the specification and the appended claims of this application refers to any combination and all possible combinations of one or more associated listed items, and includes these combinations. Moreover, the disclosure with regard to any of the aspects is also relevant with regard to the other aspects of the disclosure.

Although the disclosure has been illustrated and described with respect to one or more implementations, equivalent alterations and modifications will occur to others skilled in the art upon the reading and understanding of this specification and the annexed drawings. In addition, while a particular feature of the disclosure may have been disclosed with respect to only one of several implementations, such feature may be combined with one or more other features of the other implementations as may be desired and advantageous for any given or particular application.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

What is claimed is:

1. A method for constructing a sensor surface, comprising:
providing a buffer fluid comprising at least one sensing particle;
providing a membrane comprising a plurality of pores, each of the plurality of pores has a pore size smaller than the sensing particle;
arranging the membrane in relation to the buffer fluid such that a first surface of the membrane being in fluidic contact with the buffer fluid; and
pushing the buffer fluid through the membrane to immobilize the sensing particle on the first surface of the membrane.

2. The method of claim 1,
wherein the sensing particle produces light, changes color, changes charge, produces gases, changes in size, or produces at least one measurable change in reaction to sample analytes.

3. The method of claim 1,
wherein the sensing particle comprises a whole-cell eukaryotic cell, a prokaryotic cell, a non-whole cell particle containing one or more whole-cell components, a hydrogel particle, a polystyrene particle, or a ceramic particle.

4. The method of claim 1,
wherein each of the plurality of pores comprise a pore size between 0.1 to 0.5 microns.

5. A system for constructing a sensor surface, comprising:
at least one pumping arrangement comprising a buffer fluid comprising at least one sensing particle, wherein the at least one pumping arrangement pushes the buffer fluid through an opening of the at least one pumping arrangement; and
at least one filter arrangement comprising a membrane comprising a plurality of pores, each of the plurality of pores having a pore size smaller than the sensing particle,
wherein the at least one filter arrangement is detachably attached to the opening of the at least one pumping arrangement such that a first surface of the membrane being in fluidic contact with the buffer fluid, and
wherein the membrane immobilizes the sensing particle on the first surface while the buffer fluid is pushed through the membrane by the at least one pumping arrangement.

6. The system of claim 5, wherein the system further comprises:
at least one further pumping arrangement comprising a further buffer fluid comprising at least one further sensing particle; and
at least one further filter arrangement comprising a further membrane comprising a further plurality of pores, wherein each of the further plurality of pores has a pore size smaller than the further sensing particle,
wherein the further membrane immobilizes the further sensing particle on a first surface of the further membrane while the further buffer fluid is pushed through the further membrane by the at least one further pumping arrangement.

7. The system of claim 5,
wherein each of the plurality of pores comprise a pore size between 0.1 to 0.5 microns.

8. The system of claim 5,
wherein each of the further plurality of pores comprise a pore size between 0.1 to 0.5 microns.

9. The system of claim 5,
wherein the at least one pumping arrangement or the at least one further pumping arrangement are syringes or have a syringe-like configuration.

10. The system of claim 5,
wherein the at least one filter arrangement or the at least one further filter arrangement are syringe filters or filters having components of a syringe.

11. The system of claim 5,
wherein the sensing particle comprises a whole-cell eukaryotic cell, a prokaryotic cell, a non-whole cell particle containing one or more whole-cell components, a hydrogel particle, a polystyrene particle, or a ceramic particle.

12. The system of claim 5,
wherein the sensing particle produces light, changes color, changes charge, produces gases, changes in size, or produces at least one measurable change in reaction to sample analytes.

13. A sensor comprising:
at least one filter arrangement comprising a membrane;
at least one sensing particle immobilized on a first surface of the membrane; and
a readout module arranged in relation to the first surface of the membrane,
wherein the at least one filter arrangement passes analytes onto a second surface of the membrane opposite to the first surface in order for the analytes to diffuse through the membrane, and
wherein the readout module detects a property of the sensing particle in reaction to the analytes diffused through the membrane.

14. The sensor of claim 13,
wherein the at least one filter arrangement is detachably attached to the readout module.

15. The sensor of claim 13,
wherein the readout module comprises an optical detector that detects an optical property of the sensing particle in reaction to the analytes diffused through the membrane.

16. The sensor of claim 13,
wherein the readout module comprises an electrical signal detector that detects an electrical property of the sensing particle in reaction to the analytes diffused through the membrane.

17. The sensor of claim 13,
wherein the readout module comprises a detector unit that simultaneously detects an optical property and an electrical property of the sensing particle in reaction to the analytes diffused through the membrane.

18. The sensor of claim 13,
wherein the at least one filter arrangement comprises a first channel in fluidic contact with the first surface of the membrane and a second channel in fluidic contact with the second surface of the membrane,
wherein the first channel confines the sensing particle immobilized on the first surface of the membrane or the analytes diffused through the membrane, and
wherein the second channel passes the analytes onto the second surface of the membrane for the analytes to diffuse through the membrane.

19. The sensor of claim 17,
wherein at least one of the at least one filter arrangement or the detector unit is sterilizable.

20. The sensor of claim 13,
wherein the sensing particle comprises a whole-cell eukaryotic cell, a prokaryotic cell, a non-whole cell particle containing one or more whole-cell components, a hydrogel particle, a polystyrene particle, or a ceramic particle.
